(19) United States
(12) Patent Application Publication
(10) Pub. No.: US 2018/0008645 A1
(43) Pub. Date: Jan. 11, 2018

(54) SCAFFOLDS FOR THE TReATMENT OF SPINAL CORD INJURIES AND DISEASES

(71) Applicant: Technion Research & Deveiopment Foundation Limited, Haifa (IL)

(72) Inventors: Shulamit Levenberg, Moreshet (IL); Sivan Ida Cohan-Matstiah, Atzmon-Segev (IL)

(21) Appl. No.: 15/548,211

(22) PCT Filed: Feb. 2, 2016
(86) PCT No.: PCT/IL2016/050113

§ 371 Date: Aug. 2, 2017

Related U.S. Application Data
(60) Provisional application No. 62/110,685, filed on Feb. 2, 2015.

(51) Int. Cl. A61K 35/30; A61L 27/18; A61L 27/38; A61L 27/48; A61L 27/54; C08L 67/04

(52) U.S. Cl. CPC A61K 35/30; A61L 27/3834; A61L 27/48; A61L 27/18; A61L 2430/38; C08L 67/04; A61L 27/54

(57) ABSTRACT

Methods of treating spinal cord injuries are disclosed. The method comprises implanting scaffolds comprising a protruding scaffold and a supporting scaffold, wherein at least a portion of the protruding scaffold is inserted into a lesioned area of the spinal cord so as to contact the injury or diseased site, wherein the supporting scaffold does not protrude into the injury or diseased site and is in contact with the rostral and/or caudal dura of the spinal cord.

### FIELD AND BACKGROUND OF THE INVENTION

The present invention, in some embodiments thereof, relates to a method of treating spinal cord injuries and diseases and, more particularly, but not exclusively, to specialized scaffolds for the use thereof.

Damage to the spinal cord may result in autonomic dysfunction, a loss of sensation or a loss of mobility. Such spinal cord injury (SCI) frequently is caused by trauma, tumors; ischemia, developmental disorders, neurodegenerative diseases, demyelinative diseases, transverse myelitis, vascular malformations, or other causes. The consequences of SCI depend on the specific nature of the injury and its location along the spinal cord. In addition because SCI is a dynamic process, the full extent of injury may not be apparent initially in all acute cord syndromes. Incomplete cord lesions may evolve into more complete lesions; more commonly, the injury level raises one or two spinal levels during the hours to days after the initial event. A complex cascade of pathophysiologic events accounts for this clinical deterioration.

The psychological and social impact of SCIs often is devastating. Some of the general disabling conditions associated with SCI are permanent paralysis of the limbs, chronic pain, muscular atrophy, loss of voluntary control over bladder and bowel, sexual dysfunction, and infertility.

Recent advances in neuroscience have drawn considerable attention to research into SCI and have made significantly better treatment and rehabilitation options available. Functional electrical stimulation (FES), for example, has shown the potential to enhance nerve regeneration and allow significant improvements in restoring and improving functional capacity after SCI. However, not all patients with spinal cord injury qualify for FES (a complete lesion of the spinal cord must be established); the patient must be in a neurologically stable condition; and the peripheral nerves must be intact to respond to exogenous electrical stimulations. Therefore, tissue engineering methods that could successfully restore, maintain, and improve the damage caused by spinal cord injury would eliminate many of the problems associated with current treatment options. The development of improved tissue regeneration strategies will require a multi-disciplinary approach combining several technologies. Due to the size and complexity of tissues such as the spinal cord and articular cartilage, specialized constructs incorporating cells may be a promising strategy for achieving functional recovery.

Background art includes U.S. Pat. No. 7,666,177, U.S. Patent Application No. 20040047843, U.S. Pat. No. 6,273,905, U.S. Pat. No. 8,328,857, U.S. Pat. No. 8,377,463, WO 2005039384, CN 101979106 and U.S. Pat. No. 7,695,504.

### SUMMARY OF THE INVENTION

According to an aspect of some embodiments of the present invention there is provided a method of treating a spinal cord injury or disease in a subject comprising implanting into the subject a scaffold, the scaffold comprising a protruding scaffold and a supporting scaffold, wherein at least a portion of the protruding scaffold is inserted into a lesioned area of the spinal cord so as to contact the injury or diseased site, wherein the supporting scaffold does not protrude into the injury or diseased site and is in contact with the rostral and/or caudal dura of the spinal cord, wherein the supporting scaffold and the protruding scaffold are in physical contact with one another following the implanting and the supporting scaffold is orientated with respect to the protruding scaffold to form a shape comprising a T following the implanting, thereby treating the spinal cord injury or disease.

According to an aspect of some embodiments of the present invention there is provided an article of manufacture comprising a first scaffold and a second scaffold wherein:
(i) the first scaffold is seeded with cells and is of dimensions such that it is capable of protruding into a lesioned area of the spinal cord of a subject; and
(ii) the second scaffold is not seeded with cells.

According to an aspect of some embodiments of the present invention there is provided an article of manufacture comprising a T shaped or H shaped-scaffold, wherein the vertical portion of the T, or the horizontal portion of the H is of dimensions that that it is capable of protruding into a lesioned area of the spinal cord of a subject.

According to some embodiments of the invention, the protruding scaffold and the supporting scaffold are part of a single element.

According to some embodiments of the invention, the protruding scaffold is a separate element to the supporting scaffold.

According to some embodiments of the invention, the protruding scaffold is implanted prior to the supporting scaffold.

According to some embodiments of the invention, the protruding scaffold is carved into a shape of the lesioned area of the spinal cord.

According to some embodiments of the invention, the method further comprises pre-seeding the protruding scaffold with cells.

According to some embodiments of the invention, the method further comprises pre-seeding the supporting scaffold with cells.

According to some embodiments of the invention, the protruding scaffold comprises a therapeutic agent.

According to some embodiments of the invention, the supporting scaffold comprises a therapeutic agent.

According to some embodiments of the invention, the therapeutic agent is at least one agent is for promoting cell adhesion, colonization, proliferation, differentiation, extravasation and/or migration.

According to some embodiments of the invention, the therapeutic agent is selected from the group consisting of an amino acid, a small molecule chemical, a peptide, a polypeptide, a protein, a DNA, a RNA, a lipid and/or a proteoglycan.

According to some embodiments of the invention, the protein is selected from the group consisting of an extracellular matrix protein, a cell adhesion protein, a growth factor, a cytokine, a hormone, a protease and a protease substrate.

According to some embodiments of the invention, the cells are mixed with fibrin prior to the pre-seeding.

According to some embodiments of the invention, the therapeutic agent is attached to, embedded or impregnated in the scaffold.

According to some embodiments of the invention, the lesioned area comprises cysts.

According to some embodiments of the invention, the protruding scaffold and the supporting scaffold are fabricated from an identical material.

According to some embodiments of the invention, the protruding scaffold and the supporting scaffold are fabricated from a non-identical material.

According to some embodiments of the invention, the material is a biodegradable porous material.

According to some embodiments of the invention, the material is synthetic.

According to some embodiments of the invention, the material is non-synthetic.

According to some embodiments of the invention, the material is selected from the group consisting of poly(L-lactic acid), poly(lactic acid-co-glycolic acid), collagen-GAG, collagen, fibrin, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).

According to some embodiments of the invention, the material comprises poly(L-lactic acid) and poly(lactic acid-co-glycolic acid).

According to some embodiments of the invention, the average pore diameter of the porous material is between 300-600 μm.

According to some embodiments of the invention, the cells comprise stem cells.

According to some embodiments of the invention, the cells have been ex vivo differentiated from stem cells into a neuronal lineage.

According to some embodiments of the invention, the cells comprise olfactory bulb cells.

According to some embodiments of the invention, the first scaffold is carved into a shape of the lesioned area of the spinal cord.

According to some embodiments of the invention, the vertical portion is carved into a shape of the lesioned area of the spinal cord.

According to some embodiments of the invention, the scaffold is pre-seeded with cells.

According to some embodiments of the invention, the first scaffold and/or the second scaffold comprises a therapeutic agent.

According to some embodiments of the invention, the scaffold comprises a therapeutic agent.

According to some embodiments of the invention, the therapeutic agent is at least one agent is for promoting cell adhesion, colonization, proliferation, differentiation, extravasation and/or migration.

According to some embodiments of the invention, the therapeutic agent is selected from the group consisting of an amino acid, a small molecule chemical, a peptide, a polypeptide, a protein, a DNA, an RNA, a lipid and/or a proteoglycan.

According to some embodiments of the invention, the protein is selected from the group consisting of an extracellular matrix protein, a cell adhesion protein, a growth factor, a cytokine, a hormone, a protease and a protease substrate.

According to some embodiments of the invention, the cell adhesion protein is fibrin.

According to some embodiments of the invention, the therapeutic agent is attached to, embedded or impregnated in the scaffold.

According to some embodiments of the invention, the first scaffold and the second scaffold are fabricated from an identical material.

According to some embodiments of the invention, the first scaffold and the second scaffold are fabricated from a non-identical material.

According to some embodiments of the invention, the material is a biodegradable porous material.

According to some embodiments of the invention, the scaffold is fabricated from a biodegradable porous material.

According to some embodiments of the invention, the material is synthetic.

According to some embodiments of the invention, the material is non-synthetic.

According to some embodiments of the invention, the material is selected from the group consisting of poly(L-lactic acid), poly(lactic acid-co-glycolic acid), collagen-GAG, collagen, fibrin, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).

According to some embodiments of the invention, the material comprises poly(L-lactic acid) and poly(lactic acid-co-glycolic acid).

According to some embodiments of the invention, the average pore diameter of the porous material is between 300-600 μm.

According to some embodiments of the invention, the scaffold is seeded with cells.

According to some embodiments of the invention, the cells comprise stem cells.

According to some embodiments of the invention, the cells have been ex vivo differentiated from stem cells into a neuronal lineage.

According to some embodiments of the invention, the cells comprise olfactory bulb cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D. Dual scaffold implantation. (A) Schematic presentation of dual scaffold implantation. (B) Vertebrate laminectomy is performed to expose the spinal cord. In this procedure, the spinal lamina (L) is removed in order to access the spinal cord (SC). (C) The spinal cord is completely transected and an inner scaffold (IS) is implanted between the spinal cord stumps. (D) A sealing scaffold (US) is positioned on top of the spinal cord stumps and inner scaffold, secured below the spinal muscles (SM) by sutures.

FIG. 2 is an illustration of a single T-shaped scaffold according to embodiments described herein.

FIG. 3 is an illustration of two scaffolds which can make a T shape following implantation according to embodiments described herein.

FIG. 4A illustrates the positioning of the scaffolds described herein following implantation.

FIG. 4B illustrates an exemplary penetrating scaffold according to embodiments described herein.

### DESCRIPTION OF SPECIFIC EMBODIMENTS OF THE INVENTION

The present invention, in some embodiments thereof, relates to a method of treating spinal cord injuries and diseases and, more particularly, but not exclusively, to specialized scaffolds which comprise a T shape for the use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Spinal cord injuries have devastating impacts. Injured patients suffer from both loss of motor and sensory function. Clinically, spinal cord injuries result in functional deficits due to damage to axons, loss of neurons and glia, and demyelination. In addition, spinal cord injuries trigger secondary processes including ischemia, anoxia, and excitotoxicity. Following injury, axons of the central nervous system (CNS) consistently fail to regenerate and reinnervate central targets. Despite significant efforts in the field of grafts, transplants and methods designed to provide therapeutics, nutrients, cells and other factors to the injury site, there is currently no effective treatment for spinal cord injury.

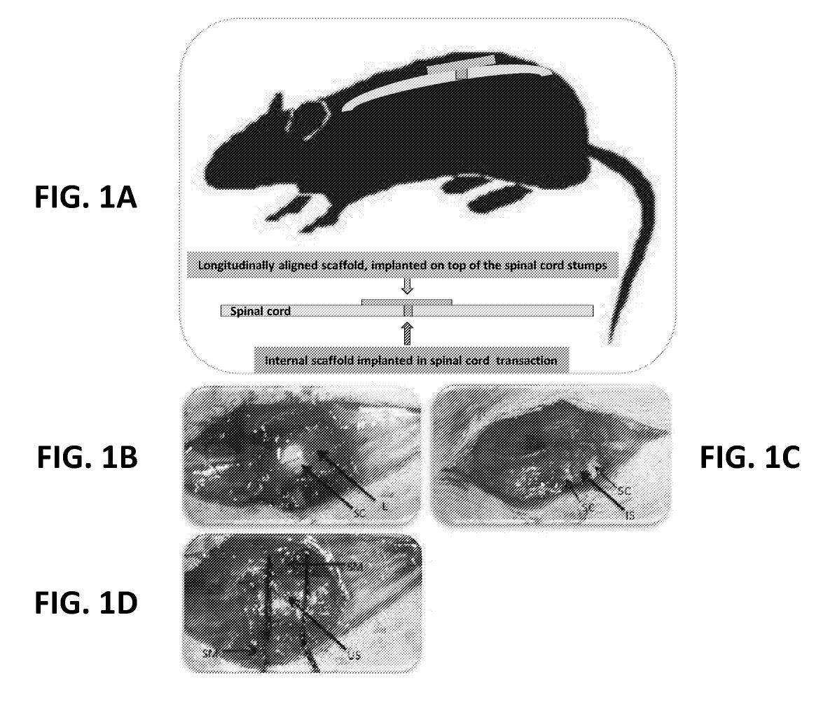

The present invention relates generally to the treatment of spinal cord injury by providing a method for delivery of cells, factors or substances to the injury area while maintaining proper conditions for the severed spinal cord. The present inventors have shown that scaffolds which comprise T shapes, whereby the vertical section of the T is inserted into the injury site and the horizontal section of the T covers and protects the injury site is advantageous over non T-shaped scaffolds (see FIGS. 1A-D). The horizontal section of the T shaped scaffold seals the injury site, supports healing of the meninges, directs sprouting of neurons and/or provides physical support for the severed spinal cord.

Thus, according to one aspect of the present invention there is provided a method of treating a spinal cord injury or disease in a subject comprising implanting into the subject a scaffold, the scaffold comprising a protruding scaffold and a supporting scaffold, wherein at least a portion of the protruding scaffold is inserted into a lesioned area of the spinal cord so as to contact the injury or diseased site, wherein the supporting scaffold does not protrude into the injury or diseased site and is in contact with the rostral and/or caudal dura of the spinal cord, wherein the supporting scaffold and the protruding scaffold are in physical contact with one another following the implanting and the supporting scaffold is orientated with respect to the protruding scaffold to form a shape comprising a T following the implanting, thereby treating the spinal cord injury or disease.

As used herein, the phrase “spinal cord injury” refers to an injury to the spinal cord that is caused by trauma instead of disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. The abbreviation “SCI” means spinal cord injury.

The spinal cord injury may be susceptible to secondary tissue injury, including but not limited to: glial scarring, myelin inhibition, demyelination, cell death, lack of neurotrophic support, ischemia, free-radical formation, and excitotoxicity.

Diseases of the spinal cord include but are not limited to autoimmune diseases (e.g. multiple sclerosis), inflammatory diseases (e.g. Arachnoiditis), neurodegenerative diseases, polio, spinabifida and spinal tumors.

The spinal cord injury may be an acute or chronic injury.

As used herein, the term “scaffold” refers to a three dimensional structure comprising a biocompatible material that provides a surface suitable for adherence and proliferation of cells. A scaffold may further provide mechanical stability and support.

It will be appreciated that the scaffold may be implanted as a single unit or as a plurality of units. When implanted as a single unit, the scaffold itself has a shape which comprises a T. Thus, the scaffold may be a T shaped scaffold or an H shaped scaffold. When implanted as a plurality of separate units, each individual unit may be of any shape (e.g. cylinders, blocks etc) as long as when they are implanted they comprise a T shape.

It will be appreciated that the two arms of the T (i.e. the vertical arm and the horizontal arm) typically cross at right angles, although it will be appreciated that the angle may also be 99°, 98°, 97°, 96°, 55°, 94°, 93°, 92°, 91°, 89°, 88°, 87°, 86°, 85°, 84°, 83°, 82°, 81° or 80°.

In a preferred embodiment the horizontal arm of the T extends equally from both sides of the vertical arm.

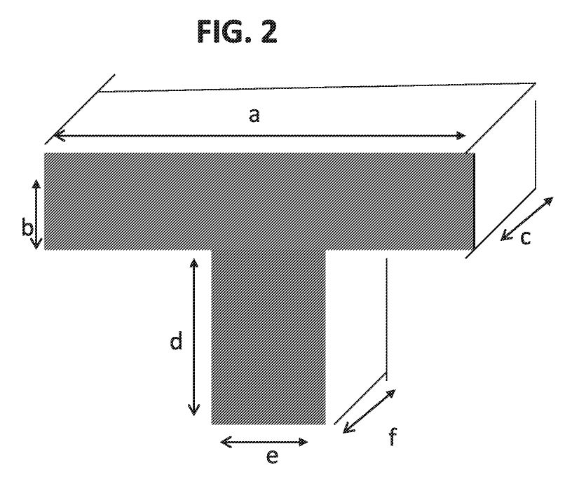

Referring now to FIG. 2, which illustrates a single scaffold having a T shape. The horizontal section of the scaffold is referred to herein as the supporting section of the scaffold and the vertical section of the scaffold is referred to herein as the protruding section of the scaffold.

A thin, elongated cylinder is one possible configuration for the protruding section and/or horizontal section, but other shapes, such as elongated rectangular tubes, spheres, helical structures, and others are possible.

The dimensions of the scaffold will vary accordingly with the spinal cord lesion to be treated. For example, the length of the protruding section can be smaller than or substantially the same size as the depth of the lesion to be treated.

It will be further appreciated that the dimensions of the scaffold will vary according to the size of the subject. Thus, the dimensions of a scaffold for treating humans will be approximately ten or even twenty times greater than the dimensions of a scaffold for treating a small animal (e.g. rodent).

For a human, the height “d” of the protruding section, as illustrated in FIG. 2 is typically between 0.1 cm-3 cm, for example between 0.5 cm-3 cm, 0.5 cm-2 cm or 2-3 cm. For a rectangular protruding section, “e” may be between 0.1-2 cm, more preferably between 0.1-1 cm, more preferably between 0.1-0.5 cm and “f” may be between 0.1-2 cm, more preferably between 0.5-2 cm, more preferably between 0.5-1 cm. For a cylindrical protruding section, the diameter of the cylinder may be between 0.1-2 cm, more preferably between 0.5-2 cm, more preferably between 0.5-1 cm.

It will be appreciated that the protruding section may also be fashioned such that its shape mirrors the shape of the lesion to be treated.

The length of the supporting section “a” is typically between 2-10 cm, more preferably between 3-8 cm and even more preferably between 5-7 cm. The thickness “c” of the supporting section is typically between 0.5 cm-2 cm or 0.1 cm-1 cm. According to one embodiment, the thickness “c” of the supporting section is greater than the thickness “f” of the protruding section. For example the ratio of c:f may be about 1.5: 1, 2:1, 3:1 or greater.

According to a preferred embodiment, the ratio a:e is greater than 2:1, 3:1, 4:1, 5:1, 10:1 or even 20:1.

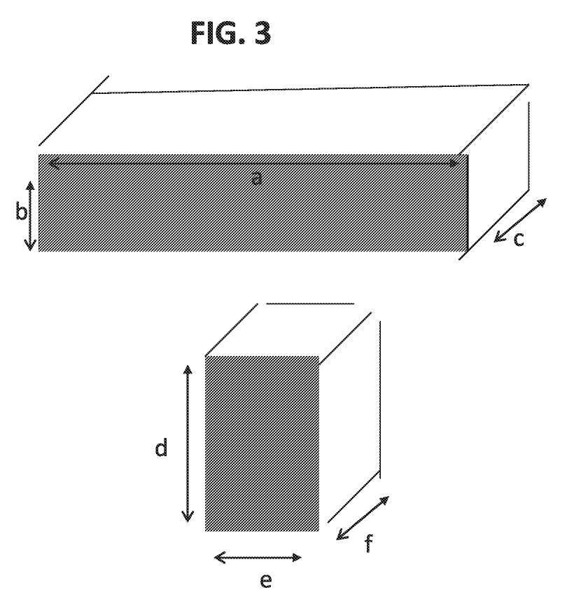

Referring now to FIG. 3, which illustrates two scaffolds which, following implantation, are capable of making a shape comprising a T shape. The scaffold which would be placed directly into the lesion is referred to herein as the protruding scaffold and is analogous to the protruding section of the scaffold described in FIG. 2 and the scaffold which would be placed on top of the protruding scaffold to generate the T shape is referred to herein as the supporting scaffold and is analogous to the supporting section of the scaffold described in FIG. 2.

A thin, elongated cylinder is one possible configuration for the protruding scaffold and/or horizontal scaffold, but other shapes, such as elongated rectangular tubes, spheres, helical structures, and others are possible.

The dimensions of the scaffolds will vary accordingly with the spinal cord lesion to be treated. For example, the length of the protruding scaffold can be smaller than or substantially the same size as the depth of the lesion to be treated.

It will be further appreciated that the dimensions of the scaffolds will vary according to the size of the subject. Thus, the dimensions of scaffolds for treating humans will be approximately ten or even twenty times greater than the dimensions of scaffolds for treating a small animal (e.g. rodent).

For a human, the height “d” of the protruding scaffold, as illustrated in FIG. 2 is typically between 0.1 cm-3 cm, for example between 0.5 cm-3 cm, 0.5 cm-2 cm or 2-3 cm. For a rectangular protruding section, “e” may be between 0.1-2 cm, more preferably between 0.1-1 cm, more preferably between 0.1-0.5 cm and “f” may be between 0.1-2 cm, more preferably between 0.5-2 cm, more preferably between 0.5-1 cm. For a cylindrical protruding section, the diameter of the cylinder may be between 0.1-2 cm, more preferably between 0.5-2 cm, more preferably between 0.5-1 cm.

It will be appreciated that the protruding scaffold may also be fashioned such that its shape mirrors the shape of the lesion to be treated.

The length of the supporting scaffold “a” is typically between 2-10 cm, more preferably between 3-8 cm and even more preferably between 5-7 cm. The thickness “c” of the supporting scaffold is typically between 0.5 cm-2 cm or 0.1 cm-1 cm. According to one embodiment, the thickness “c” of the supporting scaffold is greater than the thickness “f” of the protruding scaffold. For example the ratio of c:f may be about 1.5: 1, 2:1, 3:1 or greater.

According to a preferred embodiment, the ratio a:e is greater than 2:1, 3:1, 4:1, 5: 1, 10:1 or even 20:1.

The scaffolds of the present invention may be made uniformly of a single polymer, co-polymer or blend thereof. However, it is also possible to form a scaffold according to the invention of a plurality of different polymers. There are no particular limitations to the number or arrangement of polymers used in forming the scaffold. Any combination which is biocompatible, may be formed into fibers, and degrades at a suitable rate, may be used.

Both the choice of polymer and the ratio of polymers in a co-polymer may be adjusted to optimize the stiffness of the scaffold. The molecular weight and cross-link density of the scaffold may also be regulated to control both the mechanical properties of the scaffold and the degradation rate (for degradable scaffolds). The mechanical properties may also be optimized to mimic those of the tissue at the implant site.

Scaffold material may comprise natural or synthetic organic polymers that can be gelled, or polymerized or solidified (e.g., by aggregation, coagulation, hydrophobic interactions, or cross-linking) into a 3-D open-lattice structure that entraps water or other molecules, e.g., to form a hydrogel. Structural scaffold materials may comprise a single polymer or a mixture of two or more polymers in a single composition. Additionally, two or more structural scaffold materials may be co-deposited so as to form a polymeric mixture at the site of deposition. Polymers used in scaffold material compositions may be biocompatible, biodegradable and/or bioerodible and may act as adhesive substrates for cells. In exemplary embodiments, structural scaffold materials are easy to process into complex shapes and have a rigidity and mechanical strength suitable to maintain the desired shape under in vivo conditions.

In certain embodiments, the structural scaffold materials may be non-resorbing or non-biodegradable polymers or materials.

The phrase “non-biodegradable polymer”, as used herein, refers to a polymer or polymers which at least substantially (i.e. more than 50%) do not degrade or erode in vivo. The terms “non-biodegradable” and “non-resorbing” are equivalent and are used interchangeably herein.

Such non-resorbing scaffold materials may be used to fabricate materials which are designed for long term or permanent implantation into a host organism. In exemplary embodiments, non-biodegradable structural scaffold materials may be biocompatible. Examples of biocompatible non-biodegradable polymers which are useful as scaffold materials include, but are not limited to, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art.

In other embodiments, the structural scaffold materials may be a “bioerodible” or “biodegradable” polymer or material.

The phrase “biodegradable polymer” as used herein, refers to a polymer or polymers which degrade in vivo, and wherein erosion of the polymer or polymers over time occurs concurrent with or subsequent to release of the islets. The terms “biodegradable” and “bioerodible” are equivalent and are used interchangeably herein.

Such bioerodible or biodegradable scaffold materials may be used to fabricate temporary structures. In exemplary embodiments, biodegradable or bioerodible structural scaffold materials may be biocompatible. Examples of biocompatible biodegradable polymers which are useful as scaffold materials include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers. In still other embodiments, a mixture of non-biodegradable and bioerodible and/or biodegradable scaffold materials may be used to form a biomimetic structure of which part is permanent and part is temporary.

PLA, PGA and PLA/PGA copolymers are particularly useful for forming the scaffolds of the present invention. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester scaffold is related to the molecular weights. The higher molecular weights, weight average molecular weights of 90,000 or higher, result in polymer scaffolds which retain their structural integrity for longer periods of time; while lower molecular weights, weight average molecular weights of 30,000 or less, result in both slower release and shorter scaffold lives. For example, poly(lactide-co-glycolide) (50:50) degrades in about six weeks following implantation.

According to a preferred embodiment of this aspect of the present invention the scaffold comprises a 50:50 blend of (1) poly(lactic-co-glycolic acid) and (2) poly-L-lactic acid (PLLA). It is preferred that any of the foregoing articles have a degradation rate of about between about 30 and 90 days 9 (e.g. about 6 weeks, 7 weeks, eight weeks, nine week or ten weeks); however, the rate can be altered to provide a desired level of efficacy of treatment.

The molecular weight (MW) of the polymers used to fabricate the presently described scaffolds can vary according to the polymers used and the degradation rate desired to be achieved. In one embodiment, the average MW of the polymers in the scaffold is between about 1,000 and about 50,000. In another embodiment, the average MW of the polymers in the scaffold is between about 2,000 and 30,000. In yet another embodiment, the average MW is between about 20,000 and 50,000 for PLGA and between about 300,000 and 500,000 for PLLA.

Advantageously, the polymeric material may be fabricated as a putty. By “putty” it is meant that the material has a dough-like consistency that is formable or moldable. These materials are sufficiently and readily moldable such that they can be carved into flexible three-dimensional structures or shapes complementary to a target site to be treated.

In certain embodiments, the structural scaffold material composition is solidified or set upon exposure to a certain temperature; by interaction with ions, e.g., copper, calcium, aluminum, magnesium, strontium, barium, tin, and di-, tri- or tetra-functional organic cations, low molecular weight dicarboxylate ions, sulfate ions, and carbonate ions; upon a change in pH; or upon exposure to radiation, e.g., ultraviolet or visible light. In an exemplary embodiment, the structural scaffold material is set or solidified upon exposure to the body temperature of a mammal, e.g., a human being. The scaffold material composition can be further stabilized by cross-linking with a polyion.

In an exemplary embodiment, scaffold materials may comprise naturally occurring substances, such as, fibrinogen, fibrin, thrombin, chitosan, collagen, alginate, poly(N-isopropylacrylamide), hyaluronate, albumin, collagen, synthetic polyamino acids, prolamines, polysaccharides such as alginate, heparin, and other naturally occurring biodegradable polymers of sugar units.

In certain embodiments, structural scaffold materials may be ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix. In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole). Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Bioerodible or biodegradable polymers, i.e., polymers that dissolve or degrade within a period that is acceptable in the desired application (usually in vivo therapy), will degrade in less than about five years or in less than about one year, once exposed to a physiological solution of pH 6-8 having a temperature of between about 25.degree. C. and 38.degree. C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Typically, the scaffolds of the present invention are porous. The porosity of the scaffold may be controlled by a variety of techniques known to those skilled in the art. The minimum pore size and degree of porosity is dictated by the need to provide enough room for the cells and for nutrients to filter through the scaffold to the cells. The maximum pore size and porosity is limited by the ability of the scaffold to maintain its mechanical stability after seeding. As the porosity is increased, use of polymers having a higher modulus, addition of stiffer polymers as a co-polymer or mixture, or an increase in the cross-link density of the polymer may all be used to increase the stability of the scaffold with respect to cellular contraction.

According to a preferred embodiment of this aspect of the present invention, the scaffold has an average pore diameter of about 300-600 μm.

Electrical signals in the form of action potentials are the means of signaling for billions of cells in the central nervous system. Numerous studies have shown that this electrical activity is not only a means of communication, but also necessary for the normal development of the nervous system and refinement of functional neural circuits. In the case of spinal cord injury, cell-to-cell communication may be interrupted and the mechanisms of normal neurological development imply that electrical activity should be part of the restoration of functional connections. Such activity is important for the survival of existing cells and the incorporation of any transplanted cells into working circuits. In an embodiment of the present invention, the scaffolds are fabricated from synthetic biomaterials and are capable of conducting electricity and naturally eroding inside the body. In an exemplary embodiment, the scaffolds comprise a biocompatible polymer capable of conducting electricity e.g. a polypyrrole polymer. Polyaniline, polyacetyline, poly-p-phenylene, poly-p-phenylene-vinylene, polythiophene, and hemosin are examples of other biocompatible polymers that are capable of conducting electricity and may be used in conjunction with the present invention. Other erodible, conducting polymers are well known (for example, see Zelikin et al., Erodible Conducting Polymers for Potential Biomedical Applications, Angew. Chem. Int. Ed. Engl., 2002, 41(1):141-144). Any of the foregoing electrical conducting polymers can be applied or coated onto a malleable or moldable scaffold.

The scaffolds may be made by any of a variety of techniques known to those skilled in the art. Salt-leaching, porogens, solid-liquid phase separation (sometimes termed freeze-drying), and phase inversion fabrication may all be used to produce porous scaffolds. Fiber pulling and weaving (see, e.g. Vacanti, et al., (1988) Journal of Pediatric Surgery, 23: 3-9) may be used to produce scaffolds having more aligned polymer threads. Those skilled in the art will recognize that standard polymer processing techniques may be exploited to create polymer scaffolds having a variety of porosities and microstructures.

Scaffold materials are readily available to one of ordinary skill in the art, usually in the form of a solution (suppliers are, for example, BDH, United Kingdom, and Pronova Biomedical Technology a.s. Norway). For a general overview of the selection and preparation of scaffolding materials, see the American National Standards Institute publication No. F2064-00 entitled Standard Guide for Characterization and Testing of Alginates as Starting Materials Intended for Use in Biomedical and Tissue Engineering Medical Products Applications”.

Therapeutic compounds or agents that modify cellular activity can also be incorporated (e.g. attached to, coated on, embedded or impregnated) into the scaffold material. Campbell et al (US Patent Application No. 20030125410) which is incorporated by reference as if fully set forth by reference herein, discloses methods for fabrication of 3D scaffolds for stem cell growth, the scaffolds having preformed gradients of therapeutic compounds. The scaffold materials, according to Campbell et al, fall within the category of “bio-inks”. Such “bio-inks” are suitable for use with the compositions and methods of the present invention.

Exemplary agents that may be incorporated into the scaffold of the present invention include, but are not limited to those that promote cell adhesion (e.g. fibronectin, integrins), cell colonization, cell proliferation, cell differentiation, anti-inflammatories, cell extravasation and/or cell migration. Thus, for example, the agent may be an amino acid, a small molecule chemical, a peptide, a polypeptide, a protein, a DNA, an RNA, a lipid and/or a proteoglycan.

Proteins that may be incorporated into the scaffolds of the present invention include, but are not limited to extracellular matrix proteins, cell adhesion proteins, growth factors, cytokines, hormones, proteases and protease substrates. Thus, exemplary proteins include vascular endothelial-derived growth factor (VEGF), activin-A, retinoic acid, epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, erythropoietin, interleukins, tumor necrosis factors, interferons, colony stimulating factors, basic and acidic fibroblast growth factors, nerve growth factor (NGF) or muscle morphogenic factor (MMP). The particular growth factor employed should be appropriate to the desired cell activity. The regulatory effects of a large family of growth factors are well known to those skilled in the art.

The protruding scaffold (and optionally the supporting scaffold) is typically seeded with cells prior to implantation. The cells in the protruding scaffold and supporting scaffold may be identical or non-identical. Due to the size of the supporting scaffold, typically the ratio of cells in the supporting scaffold is greater than 2:1, 3:1 or even 4:1.

The cells may be stem cells such as mesenchymal stem cells, neuronal stem cells or embryonic stem cells. The stem cells may be manipulated ex vivo so that they are differentiated partially or fully into cells of the neuronal lineage (e.g. neurons, astrocytes, oligodendrocytes) and/or are capable of secreting trophic factors (e.g. neurotrophic factors such as BDNF, GDNF etc.). Other cells contemplated by the present inventors are olfactory bulb cells. Ex vivo differentiation of the cells may be effected prior to scaffold seeding or following scaffold seeding.

Methods of differentiating mesenchymal stem cells into cells of the neuronal lineage are provided for example in WO2006/134602, WO2009/144718, WO2007/066338 and WO2004/046348, the teachings of which are incorporated herein by reference.

The cells may be genetically modified or non-genetically modified. For example, the cells may be genetically modified to express an exogenous polypeptide or polynucleotide (e.g. an RNA silencing agent such as siRNA).

According to a particular embodiment, the cells are human.

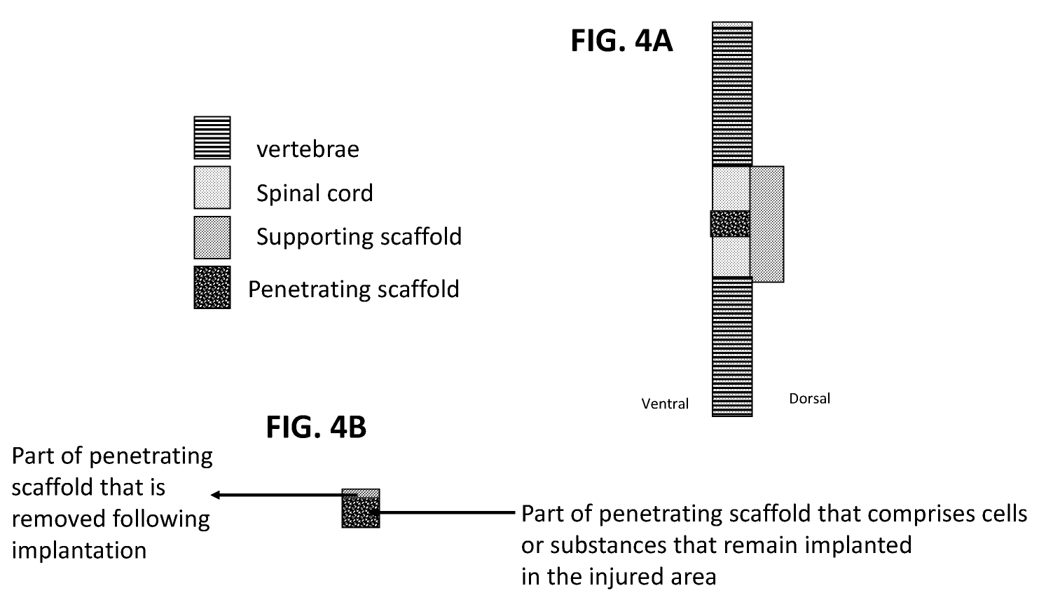

According to a particular embodiment, a portion of the penetrating scaffold is seeded with cells and a portion of the penetrating scaffold is not seeded with cells. For this embodiment, the portion of the scaffold which is not seeded with cells is typically the part of the scaffold that is in contact with the implantation device (e.g. tweezers) during the implantation procedure (as illustrated in FIG. 4B). This portion of the scaffold may be removed following implantation.

Cells can be seeded in the scaffold by static loading, or, more preferably, by seeding in stirred flask bioreactors (scaffold is typically suspended from a solid support), in a rotating wall vessel, or using direct perfusion of the cells in medium in a bioreactor. Highest cell density throughout the scaffold is achieved by the latter (direct perfusion) technique.

The cells may be seeded directly onto the scaffold, or alternatively, the cells may be mixed with a gel which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components. An exemplary gel is Matrigel™, from Becton-Dickinson. Matrigel™ is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J. Cell. Biochem. 40:215, 1989), but it does not contain any detectable levels of tissue inhibitors of metalloproteinases (TIMPs) (Mackay, A. R., et al., BioTechniques 15:1048, 1993). Alternatively, the gel may be growth-factor reduced Matrigel, produced by removing most of the growth factors from the gel (see Taub, et al., Proc. Natl. Acad. Sci. USA (1990); 87 (10:4002-6). In another embodiment, the gel may be a collagen I gel, alginate, or agar. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

According to a particular embodiment, the gel comprises fibrin.

For treating spinal cord injuries (e.g. a compression spinal cord injury), the protruding scaffold (or protruding section of the single scaffold) is implanted directly into the wound (e.g. into the epicenter of the injury), wherein the scaffold runs through the injury site as illustrated in FIG. 4A. The scaffold can be inserted through a surgical incision directly into the lesion to be treated.

Following implantation of the prortruding scaffold, the supporting scaffold is implanted. The supporting scaffold extends beyond the caudal and rostral sides of the injured site and preferably at a distance of approximately ¼ or ½ the length of the injured site. In a preferred embodiment supporting scaffold will extend equally beyond the caudal and rostral sides of the injured.

The supporting scaffold does not protrude into the injury or diseased site and is in contact with the rostral and/or caudal dura of the spinal cord. Further, the supporting scaffold is implanted such that it is in direct contact with the penetrating scaffold—see FIG. 4A. Following implantation of the supporting scaffold, the muscle layer above is sutured such that it presses against the area of the spinal cord and greatly reduces the movement of the spinal cord. By constraining the spinal cord in this way, and reducing movement, glial scar formation is reduced.

It is expected that during the life of a patent maturing from this application many relevant scaffolds will be developed and the scope of the term scaffold is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### Surgical Procedures for Spinal Cord Transection

Materials and Methods

All procedures were carried out under the supervision of the Institutional Animal Care and Use Committee and according to the Technion Guide for the Care and Use of Laboratory Animals.

Cells: Syngeneic cells from the outer layers of postnatal day-7 (P7) Wistar rats OBCs (nerve fiber and glomerular layers) were isolated into ice-cold PBS. Then OBCs were washed twice in PBS, chopped with a scalpel and trypsinized. The cells were filtered through a 23 gauge needle to generate single-cell suspension. Cells were cultured in 2D flasks for 14-17 days, in DMEM/F12 Nutrient Mix+10% fetal bovine serum and 1:100 Insulin transferrin selenium-X.

Poly-L-lactic acid (PLLA)/Poly lactic coglicolic acid (PLGA) dual scaffold implantation (inner and sealing scaffolds): Animals were anesthetized by intraperitoneal injection of ketamine (80 mg/kg) and xylazine (10 mg/kg), or Isoflurane (3%) followed by buprenorphine (0.01 mg/kg). Ophthalmic ointment was applied to the eyes. Fur was shaved and povidon-iodine 10% was applied to the skin. The skin and the muscles of the back were cut longitudinally and laminectomy was performed at the T10 segment. Post-laminectomy, a complete transection of the spinal cord was performed using micro-scissors. A 3 mm×3 mm×1 mm PLLA\PLGA scaffold with or without cells was implanted into the lesion site as illustrated in FIGS. 1A-D. When needed, size adjustments were performed using micro-scissors. Spinal cord stumps were tightened against the scaffold. A sealing scaffold (˜100 mm×3 mm×1 mm) was placed to cover the injured and healthy spinal cord tissue at the rostral and the caudal aspects of the lesion. Then the muscle and skin were closed with sutures.

Injection of cells without scaffolds: Cells were cultured in a flask. Media used were identical to those applied to the cells on a scaffold. Prior to the surgery, the cells (0.5×10⁶) suspended in expansion medium were centrifuged. Fibrin-based constructs were prepared by mixing cellular preparations with 5 μl human thrombin (50/ml, Sigma) inside a vial. The same amount of fibrinogen solution (15 mg/ml, Sigma) was then added and the mixture was gently pipetted. Post laminectomy, a complete transection of the spinal cord was performed using micro-scissors. The cellular preparation mixed with fibrin was applied using a pipette into the injury site, uniformly filling the gap between the stumps of the spinal cord. After being left to polymerize for 3 minutes, the muscle and the skin were sutured.

Results

OBCs with dual scaffold yielded a group of walking rats (more than 20% regained walking capability BBB>14). In contrast, rats that were transplanted with OBCs in the absence of a scaffold failed to regain walking capabilities.

In contrast, rats transplanted with cells seeded on PLA/PLGA scaffolds (without sealing) achieved BBB score of approximately 6. This was reported with mesenchymal cells [Kkot Nim Kang, (2012), Biomaterials 33(19):4783-4974] Other factors seeded on PLGA scaffold yielded even lower BBB scores of ˜4[Fan et al., 2011, J. Biomed. Mater. Res., 97B: 271-277. doi: 10.1002/jbm.b.31810].

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a spinal cord injury or disease in a subject comprising implanting into the subject a scaffold, said scaffold comprising a protruding scaffold and a supporting scaffold, wherein at least a portion of said protruding scaffold is inserted into a lesioned area of the spinal cord so as to contact the injury or diseased site, wherein said supporting scaffold does not protrude into the injury or diseased site and is in contact with the rostral and/or caudal dura of the spinal cord, wherein said supporting scaffold and said protruding scaffold are in physical contact with one another following said implanting and said supporting scaffold is orientated with respect to said protruding scaffold to forma shape comprising a T following said implanting, thereby treating the spinal cord injury or disease.

2. The method of claim 1, wherein said protruding scaffold and said supporting scaffold are part of a single element.

3. The method of claim 1, wherein said protruding scaffold is a separate element to said supporting scaffold.

4. (canceled)

5. The method of claim 1, wherein said protruding scaffold is carved into a shape of said lesioned area of the spinal cord.

6. The method of claim 1, further comprising pre-seeding said scaffold with cells.

7-15. (canceled)

16. The method of claim 1, wherein said protruding scaffold and said supporting scaffold are fabricated from an identical material.

17. (canceled)

18. The method of claim 16, wherein said material is a biodegradable porous material.

19. The method of claim 16, wherein said material is synthetic.

20-21. (canceled)

22. The method of claim 19, wherein said material comprises poly(L-lactic acid) and polylactic acid-co-glycolic acid).

23-25. (canceled)

26. The method of claim 6, wherein said cells comprise olfactory bulb cells,

27. An article of manufacture comprising a first scaffold and a second scaffold wherein:
(i) said first scaffold is seeded with cells and is of dimensions such that it is capable of protruding into a lesioned area of the spinal cord of a subject; and
(ii) said second scaffold is not seeded with cells.

28. An article of manufacture comprising a T shaped or H shaped-scaffold, wherein the vertical portion of the T, or the horizontal portion of the H is of dimensions that it is capable of protruding into a lesioned area of the spinal cord of a subject.

29-30. (canceled)

31. The article of manufacture of claim 28, wherein said scaffold is pre-seeded with cells.

32. The article of manufacture of claim 27, wherein said first scaffold and/or said second scaffold comprises a therapeutic agent.

33. The article of manufacture of claim 28, wherein said scaffold comprises a therapeutic agent.

34-38. (canceled)

39. The article of manufacture of claim 27, wherein said first scaffold and said second scaffold are fabricated from an identical material.

40-42. (canceled)

43. The article of manufacture of claim 39, wherein said material is synthetic.

44-45. (canceled)

46. The article of manufacture of claim 43, wherein said material comprises poly(L-lactic acid) and poly(lactic acid-co-glycolic acid).

47. (canceled)

48. The article of manufacture of claim 28, wherein said scaffold is seeded with cells.

49-50. (canceled)

51. The article of manufacture of claim 27, wherein said cells comprise olfactory bulb cells.
